(19) United States
(12) Patent Application Publication
(10) Pub. No.: US 2024/0191190 A1
(43) Pub. Date: Jun. 13, 2024

(54) PERFUSION CULTURE METHOD FOR CAR-T CELLS

(71) Applicant: JUVENTAS CELL THERAPY LTD., Tianjin (CN)

(72) Inventors: Lin SHI, Tianjin (CN); Zhiming XIE, Tianjin (CN); Xiaoyan YANG, Tianjin (CN); Xia JIN, Tianjin (CN); Haoliang TIAN, Tianjin (CN); Huan MENG, Tianjin (CN); Huijuan ZHU, Tianjin (CN); Linsheng HUANG, Tianjin (CN); Xinling LI, Tianjin (CN)

(21) Appl. No.: 18/550,750

(22) PCT Filed: Mar. 15, 2022
(86) PCT No.: PCT/CN2022/080811

§ 371 Date: Sep. 15, 2023

(30) Foreign Application Priority Data
Mar. 16, 2021 (CN) 202110278284.0

(51) Int. Cl. C12N 5/0783

(52) U.S. Cl. CPC C12N 5/0636; C12N 2500/98

(57) ABSTRACT

Provided is a perfusion culture method for CAR-T cells. The method comprises the following steps: 1) separating peripheral blood mononuclear cells from a single blood cell of a subject, and sorting the mononuclear cells to obtain T cells; 2) carrying out activation treatment on the separated T cells by using CD3/CD28 stimulation magnetic beads; 3) infecting the activated T cells by using a lentiviral vector; 4) carrying out perfusion culture on the lentivirus-infected T cells, and harvesting CAR-T cells, wherein the composition of a serum-free culture medium, which does not contain an animal-derived component, for culturing the CAR-T cells is: AIM-V+(3-9)% ISR; and the perfusion culture comprises the following stages: the first stage: when the cell density is (0.5-1.1)×10⁶ cells/mL, the perfusion rate is A1, and/or the second stage: when the cell density is (1.1-2)×10⁶ cells/mL, the perfusion rate is A2, and the third stage: when the cell density is >2×10⁶ cells/mL, the perfusion rate is A3, and the ratio of A1 to A2 to A3 is 1:2:2.5.

### TECHNICAL FIELD

The present disclosure relates to the technical field of bioengineering, especially to a perfusion culture method for CAR-T cells.

### BACKGROUND ART

CAR-T cell (Chimeric Antigen Receptor T cell), refers to the cells produced by transferring the genetic material which carries specific antigen recognition domain and T cell activation signal into T cells through gene modification technology so that T cells could be activated by directly binding to a specific antigen on the surface of tumor cells, and directly kill tumor cells by releasing perforin, granzyme B, etc., and meanwhile, recruit human endogenous immune cells to kill tumor cells by releasing cytokines, so as to achieve the purpose of tumor treatment.

The procedure for using CAR-T cells to treat tumors includes collecting peripheral blood from patients, isolating T cells, introducing CAR into T cells, culturing in vitro and transfusing cells back into the patients. In vitro culture process it needs to expand a large number of CAR-T cells, and generally one patient needs hundreds of millions, or even billions of CAR-T cells (the larger the body, the more cells are needed), while the cost of culture medium used in CAR-T cells expansion is extremely high and causes a heavy economic burden on patients. Meanwhile, the survival rate of CAR-T cells will directly affect the clearance efficiency of CAR-T cells on cancer cells. Clinical studies have proved that the proliferation ability of CAR-T cells in peripheral blood of patients after reinfusion has a strong correlation with the therapeutic effect. In addition, reference 1 (Almeida J R, Price D A, Papagno L. Arkoub Z A, Sauce D, Bornsterin E et al. Superior control of HIV-1 replication by CD8+ T cells is reflected by their avidity, polyfunctionality, and clonal turnover. J Exp Med 2007; 204: 2473-2485) and reference 2 (Harari A, Cellerai C, Enders F B. Kostler J, Codarri L. Tapia G er al. Skewed association of polyfunctional antigen-specific CD8 T cell populations with HLA-B genptype. Proc Natl Acad Sci USA 2007; 104:16233-16238) have shown that the content of cytokines (such as IFN-γ) secreted by T cells is closely related to efficacy. Therefore, how to save the culture medium as much as possible without significantly reducing the culture effect is an urgent problem to be solved.

Reference 3 (Corey Smith et al., Ex vivo expansion of human T cells for adoptive immunotherapy using the novel Xeno-free CTS Immune Cell Serum Replacement, Clinical & Translational Immunology (2015) 4, e31; doi:10.1038/cti.2014.31) disclosed a serum-free culture medium suitable for genetically modified cells (such as T cells obtained by gene transduction mediated by lentivirus), and the present disclosure further improves the culture medium and culture method based on this reference, so as to save the culture medium as much as possible without significantly reducing the culture effect.

### SUMMARY OF THE DISCLOSURE

In view of the above, the purpose of the present disclosure is to propose a perfusion culture method for CAR-T cells, which can save culture medium and have higher economy without significantly reducing the culture effect.

For the purpose, the present disclosure provided a perfusion culture method for CAR-T cells, which comprises the following steps:
1) separating peripheral blood mononuclear cells from apheresis components of a subject, and sorting the peripheral blood mononuclear cells to obtain T cells;
2) carrying out activation treatment on the separated T cells with CD3/CD28 stimulation magnetic beads;
3) infecting the activated T cells with a lentiviral vector;
4) carrying out perfusion culture of the lentivirus-infected T cells, and harvesting CAR-T cells:
wherein a serum-free culture medium which does not contain an animal-derived component is used for culturing the CAR-T cell and the composition of the serum-free culture medium is: AIM-V+(3-9)% ISR;
the perfusion culture comprises the follow stages:
the first stage: when the cell density is (0.5-1.1)×10⁶ cells/mL, the perfusion rate is A₁, and/or
the second stage: when the cell density is (1.1-2)×10⁶ cells/mL, the perfusion rate is A₂, and
the third stage: when the cell density is >2-10⁶ cells/mL, the perfusion rate is A₃,
wherein the ratio of A₁ to A₂ to A₃ is 1:2:2.5.

In a preferred embodiment of the present disclosure, wherein the composition of the serum-free culture medium which does not contain an animal-derived component is AIM-V+(4-7)% ISR;
preferably, the composition of the serum-free culture medium which does not contain an animal-derived component is AIM-V+5% TSR.

In a preferred embodiment of the present disclosure, wherein A₁ is 0.4 bioreactor volume/day, A₂ is 0.8 bioreactor volume/day, and A₃ is 1.0 bioreactor volume/day.

In a preferred embodiment of the present disclosure, wherein in step 4), the perfusion culture is carried out when the cell density is greater than or equal to the preset value;
preferably, the preset value is (0.3-1.2)×10⁶ cells/mL;
more preferably, the preset value is (0.4-1.0)×10⁶ cells/mL;
even more preferably, the preset value is 0.5×10⁶ cells/mL.

In a preferred embodiment of the present disclosure, wherein in step 4), fed-batch culture is used before the cell density reaches the preset value, and in the process of fed-batch culture, the density of (0.3-1)×10⁶ cells/mL is used as the standard for feeding, the ventilation volume is (0.1-1) L/min, the rotational speed is (4-12) rpm, and the ventilation is compressed air plus (1-10) % CO₂.

In a preferred embodiment of the present disclosure, in step 4), before the fed-batch culture, the method comprises:
transferring the infected T cells to Xuri bioreactor for fed-batch culture after the number of infected T cells reaches (5-15)×10⁷ cells.

In a preferred embodiment of the present disclosure, wherein in the process of perfusion culture, the ventilation volume is (0.3-0.8) L/min, the rotational speed is (5-15) rpm, and the ventilation is compressed air plus (1-10)% CO₂;
preferably, in the process of perfusion culture, the ventilation volume is (0.4-0.6) L/min, the rotational speed is (8-12) rpm, and the ventilation is compressed air plus (3-6) % CO₂;
more preferably, in the process of perfusion culture, the ventilation volume is 0.5 L/min, the rotational speed is 10 rpm, and the ventilation is compressed air plus 5% CO₂.

In a preferred embodiment of the present disclosure, in step 2), the step of carrying out activation treatment on the separated T cells with CD3/CD28 stimulation magnetic beads comprises:
resuspending the isolated T cells to a final concentration of (1-2)×10⁶ cells/mL, and mixing the T cells with CD3/CD28 stimulation magnetic beads at a ratio of (0.5-10) μL of CD3/CD28 magnetic beads per 1×10⁶ T cell, then culturing at 37° C.+5% CO₂ for at least 24 h.

In a preferred embodiment of the present disclosure, the isolated T cells are resuspended with the serum-free culture medium which does not contain an animal-derived component, wherein the composition of the serum-free culture medium which does not contain an animal-derived component is: AIM-V+(3-9)% ISR;
preferably, the composition of the serum-free culture medium which does not contain an animal-derived component is: AIM-V+(4-7)% ISR;
more preferably, the composition of the serum-free culture medium which does not contain an animal-derived component is: AIM-V+5% ISR.

In a preferred embodiment of the present disclosure, in step 3), the step of infecting the activated T cells with a lentiviral vector comprises:
taking out the activated and cultured T cells, adding polybrene with a final concentration of (5-10) μg/mL and mixing, then adding lentiviral vectors slowly at a multiplicity of infection=(0.25-5); mixing and then centrifuging at (1000-3000) rpm for 0.5-2.0 hours, then culturing at 37° C.+5% CO₂ for at least 24 hours.

### BRIEF DESCRIPTION OF THE DRAWINGS

FIG. 1 shows the comparison of CAR-T cell proliferation fold in different culture systems.

FIG. 2 shows the comparison of CAR-T cell survival rate in different culture systems.

FIG. 3 shows the comparison of CAR expression in different culture systems.

FIG. 4 shows the comparison of expansion fold under different perfusion processes (perfusion rate of 400 mL-1000 mL and perfusion rate of 600 mL-1800 mL).

FIG. 5 shows the comparison of survival rate under different perfusion processes (perfusion rate of 400 mL-1000 mL and perfusion rate of 600 mL-1800 mL).

FIG. 6 shows the comparison of expansion fold under different perfusion processes (perfusion rate of 800 mL-1000 mL and perfusion rate of 1000 mL-1500 mL).

FIG. 7 shows the comparison of survival rate under different perfusion processes (perfusion rate of 800 mL-1000 mL and perfusion rate of 1000 mL-1500 mL).

### DETAILED DESCRIPTION OF THE DISCLOSURE

It is necessary to indicate that, the technical or scientific terms used in one or more examples of this specification shall have the ordinary meaning as understood by people having ordinary skill in the field which the present disclosure belongs.

The experimental methods in the following examples are all conventional methods unless otherwise specified. The raw materials of the medicaments, the reagents and the like used in the following examples are all commercially available products unless otherwise specified.

Traditional CAR-T cell culture system uses a culture system containing a serum, which includes autologous serum (or plasma), AB serum, fetal bovine serum, etc. Autologous serum (or plasma) is affected by individual differences, of which, quality is uncontrollable and the quantity is limited. AB serum is obtained from allogeneic donors with AB blood type, of which, the quality consistency is better than that of autologous serum (or plasma), and the quantity is also higher than that of autologous serum (or plasma), while the transmission of blood borne diseases still cannot be completely avoid after pathogen screening and inactivation treatment are performed for a batch of AB serum, due to the fact that the batch of AB serum is derived from multiple donors. Fetal bovine serum is derived from cattle, so there is also a risk of allergic reactions in addition to the risk of pathogen transmission for fetal bovine serum. Therefore, from the perspective of CAR-T cell product development and safe use, it is necessary to develop a serum-free culture medium which does not contain an animal-derived component for culturing the CAR-T cell.

However, the current commercially available serum-free culture system has the following problems: the proliferation ability of CAR-T cells in serum-free culture system is weak; the survival rate of CAR-T cells in serum-free culture system is lower; and the CAR expression rate of CAR-T cells in serum-free culture system is lower, etc.

The present disclosure obtains a CAR-T cell culture medium of a serum-free culture system which does not contain an animal-derived component by screening serum-free culture medium and additives from different sources, so that the proliferation, survival rate and viral infection efficiency of CAR-T cells are higher, which is comparable to or better than that of serum-containing culture system.

In addition, perfusion culture method is adopted to culture CAR-T cells in the present disclosure, and the perfusion rate of each stage in the perfusion culture process is determined, so that the perfusion culture method of the present disclosure can save culture medium and have higher economic performance without significantly reducing the culture effect.

For the purpose, the present disclosure provides a perfusion culture method for CAR-T cells, which comprises the following steps:
1) separating peripheral blood mononuclear cells from apheresis components of a subject, and sorting the peripheral blood mononuclear cells to obtain T cells;
2) carrying out activation treatment on the separated T cells with CD3/CD28 stimulation magnetic beads;
3) infecting the activated T cells with a lentiviral vector:
4) carrying out perfusion culture of the lentivirus-infected T cells, and harvesting CAR-T cells,
wherein a serum-free culture medium which does not contain an animal-derived component is used for culturing the CAR-T cell and the composition of the serum-free culture medium is: AIM-V+(3-9)% ISR;
the perfusion culture comprises the follow stages:
the first stage: when the cell density is (0.5-1.1)×10⁶ cells/mL, the perfusion rate is A₁, and/or
the second stage: when the cell density is (1.1-2)×10⁶ cells/mL, the perfusion rate is A₂, and
the third stage: when the cell density is >2×10⁶ cells/mL, the perfusion rate is A₃,
wherein the ratio of A₁ to A₂ to A₃ is 1:2:2.5.

The composition of the serum-free culture medium which does not contain an animal-derived component is:
(1) serum-free base culture medium: AIM-V;
(2) additive: CTS IMMUNE CELL SR (“ISR”);
(3) ratio: AIM-V+(3-9)% ISR; preferably, ratio: AIM-V+(4-7)% ISR; more preferably, ratio: AIM-V+5% ISR.

The present disclosure compares the culture effects (expansion fold, survival rate and CAR expression rate) for CAR-T cell between AIM-V+5% ISR culture medium, serum-containing culture medium and several other common serum-free culture medium, and the results show that AIM-V+5% ISR culture medium shows better expansion fold, survival rate and CD³⁺CAR⁺ expression. Under overall consideration, the present disclosure selects AIM-V+5% ISR culture medium as the culture medium for CAR-T cell culture.

ISR is a well-defined serum substitute which does not contain bovine-derived or other animal-derived component, and the use of this serum substitute can reduce safety risks. Both AIM-V culture medium and ISR are commercially available from ThermoFisher Corporation.

At present, the large-scale culture of CAR-T cells mainly uses fed-batch culture, and the number of cells is increased by expanding the culture volume. However, in this way, when the number of cells required is large, it may not be possible to complete the culture in a single container, which will result in differences within the batch. In addition, the metabolic waste cannot be discharged during the culture process, which will affect the culture effect for cells. Perfusion culture is a culture method in which fresh culture medium is supplemented and waste liquid is discharged at the same time. Compared with general fed-batch culture method, concentration of culture medium components in perfusion culture process changes less, which can provide a stable and favorable growth environment for cells, and cell culture effect is better, and the cells can be greatly expanded without increasing the culture volume. Therefore, perfusion culture is more suitable for CAR-T cell expansion culture stage.

An important parameter in perfusion culture process is perfusion rate. When the density of living cells in reactor changes, the nutrients obtained and the metabolites taken away for each cell change, and the perfusion rate will inevitably change. There is a very important problem that how to choose the appropriate perfusion rate according to the changes in cell density over time. The present disclosure compares multiple perfusion modes and takes culture effect and economy into consideration, and finally determines the following perfusion culture process:
the first stage: when the cell density is (0.5-1.1)×10⁶ cells/mL, the perfusion rate is A₁, and/or
the second stage: when the cell density is (1.1-2)×10⁶ cells/mL, the perfusion rate is A₂, and
the third stage: when the cell density is >2×10⁶ cells/mL, the perfusion rate is A₃,
wherein the ratio of A₁ to A₂ to A₃ is 1:2:2.5.

Preferably, A₁ is 0.4 bioreactor volume/day, A₂ is 0.8 bioreactor volume/day, and A₃ is 1.0 bioreactor volume/day. For example, when the bioreactor volume is 1000 mL, the corresponding perfusion rate A₁ is 400 mL/day, perfusion rate A₂ is 800 mL/day, and perfusion rate A₃ is 1000 mL/day.

It is necessary to indicate that, the perfusion culture process of the present disclosure emphasizes the determination of the corresponding perfusion rate according to the changing cell density. The perfusion culture process of the present disclosure does not emphasize that both the first stage and the second stage must be included at the same time, while the first stage and the second stage can exist either or simultaneously, which needs to be determined according to the growth situation of cells. Specifically, the perfusion culture process of the present disclosure may comprise the first stage and the third stage, or comprise the second stage and the third stage, or comprise the first stage, the second stage and the third stage at the same time. In the actual culture process of CAR-T cells, when the cell density is measured as (0.5-1.1)×10⁶ cells/mL, the culture will be performed at the perfusion rate A₁. Then, after a certain interval (e.g. 24 hours), if the cell density is measured as >2×10⁶ cells/mL, the culture will be performed with perfusion rate A₃ directly (e.g., the perfusion culture process in Table 1); alternatively, if the cell density is measured as (1.1-2)×10⁶ cells/mL, the culture can be performed with perfusion rate A₂ directly, while if the cell density is measured as >2>10⁶ cells/mL after a certain interval (e.g. 24 hours), the culture can be performed with perfusion rate A₃ (e.g., the perfusion culture process in Table 2).

As described in the section of background art, the survival rate of CAR-T cells will directly affect the clearance efficiency of CAR-T cells on cancer cells, and the proliferation ability (i.e. expansion fold) of CAR-T cells has a strong correlation with the therapeutic effect. In addition, the mechanism of killing tumor cells by CAR-T cells, which are transfused back into the patients after expansion and activation in vitro is as follows: after binding to specific tumor antigen, CAR-T cells directly kill tumor cells by releasing perforin, granzyme B, etc., and meanwhile, recruit human endogenous immune cells to kill tumor cells by releasing cytokines, so as to achieve the purpose of tumor treatment. Among these cytokines released by CAR-T cells, IFN-γ (interferon γ) is the cytokine which plays a major role. Previous references have shown that IFN-γ content secreted by CAR-T cells is closely related to therapeutic effect. Therefore, the present disclosure focuses on these indicators, and the experimental results show that the present disclosure can achieve efficient culture in serum-free culture system under the condition of using serum-free culture medium plus specific perfusion process, and the expansion fold, survival rate, CAR expression rate and secreted IFN-γ content of the CAR-T cells obtained by the culture are higher.

In a preferred embodiment of the present disclosure, wherein in step 4), the perfusion culture is carried out when the cell density is greater than or equal to the preset value: preferably, the preset value is (0.3-1.2)×10⁶ cells/mL: more preferably, the preset value is (0.4-1.0)×10⁶ cells/mL; even more preferably, the preset value is 0.5×10⁶ cells/mL. Preferably, when the cell density is (0.5-1.1)×10⁶ cells/mL, the perfusion rate is A₁.

The present disclosure does not limit the method of separating peripheral blood mononuclear cells (PBMC) from apheresis components of a subject, such as glucan-meglumine diatrizoate (Ficoll) density gradient centrifugation method, which can be used to separate PMBC, purity of which can be up to 95%. The principle of glucan-meglumine diatrizoate (Ficoll) density gradient centrifugation method is as follows: the specific gravity of each tangible component in blood is different; when almost isotonic ficoll-hypaque mixed solution (also known as lymphocyte stratification solution) with a specific gravity of 1.077 is used for density gradient centrifugation, various blood components will be reaggregated according to the density gradient. Plasma and platelets may suspend in the upper part of the stratified liquid owing to low density. Erythrocytes and granulocytes may sink at the bottom of the stratified liquid owing to high density. The density of PBMC is slightly lower than that of the stratified liquid, so it may locate on the interface of the stratified liquid, as a result PMBC can be obtained. The present disclosure does not limit the method of sorting T cells from peripheral blood, such as magnetic activated cell sorting, which is based on the following principle: the antigen on the cell surface can bind to specific monoclonal antibody connected with magnetic beads, and an external magnetic field is used to adsorb the cells, which are connected with magnetic beads by antibodies, so as to keep the cells stay in the magnetic field, while the cells without this antigen on the surface are not magnetic because they cannot bind to the specific monoclonal antibody connected with the magnetic beads, and cannot stay in magnetic field, so that the cells with or without the magnetic properties can be separated.

In vin) culture of T cells requires the use of CD3/CD28 antibodies to stimulate T cells to achieve functional activity. CD3/CD28 antibody-coupled magnetic beads are mainly used for isolation, activation and in vitro expansion of human T cells. The 4.5 μm superparamagnetic beads is used, which are matched to the cell size and coupled to anti-CD3 and anti-CD28 antibodies, and can provide the primary signal and co-stimulatory signal required for T cell activation and expansion. First, magnetic cell isolation with CD3/CD28 immunomagnetic beads is carried out so as to isolate and enrich CD3⁺ T cells from the resulting isolation products. After isolation, CD3⁺ T cells are cultured in the presence of magnetic beads, and the beads can provide the primary and co-stimulatory signals required for T cell activation and expansion by binding to anti-CD3 and anti-CD28 antibodies on the immunomagnetic beads. Activated T cells can produce cytokines such as IL-2 (interleukin 2), GM-CSF (granulocyte macrophage stimulating factor), IFN-γ (interferon γ) and INF-α (tumor necrosis factor α) and play the role and function of T cells.

The present disclosure does not limit the lentiviral vectors involved, and the lentiviral vectors containing nucleic acid sequences encoding CAR genes in the prior art can be used in the present disclosure.

The technical solutions provided by the present disclosure are further described in combination with specific examples. The following examples are used only to illustrate the present disclosure and do not limit the protection scope of the present disclosure.

The detection methods involved in the following examples were as follows:

### 1) Cell Survival Rate

The detection sample was mixed and 20 μl of sample was absorbed into EP tube. Then 20 μl of AOPI dye solution was absorbed into EP tube, more than 1/10 of the sample volume was absorbed with sampling gun and mixed up and down for 10 times, 20 μl of mixed liquid was absorbed and added to cell counting plate. The cell counting plate was inserted into sample slot of cell counter and confirmation was clicked. Living cells showed green or yellow-green uniform fluorescence, and dead cells showed red fluorescence. Cell survival rate and living cell concentration were recorded.

### 2) Cell Expansion Fold

According to the cell counting results and culture volume in the cell survival rate detection assay, the total number of living cells on the day was calculated, and the total number of living cells was divided by the total number of living cells counted on the day of inoculation to obtain the cell expansion fold.

Cell expansion fold=living cell concentration×volume/number of living cells inoculated

### 3) CAR Expression Rate

The number of CAR-T cells after expansion was counted, 10⁵ of transfected and un-transfected CAR-T cells were taken out and transferred into FACS tube: the cells were washed with 2 ml of FACS buffer, centrifuged at 1200 rpm/min for 5 minutes, and the supernatant was discarded: the cell precipitate was resuspended with 100 μl of FACS buffer, 2 μl PE labeled sheep anti-rat F(ab′)² antibody was added, and the cells were stained at 4° C. for 30 minutes away from light; the cells were washed with 2 ml of FACS buffer, centrifuged at 1200 rpm/min for 5 minutes, and the supernatant was discarded; the cell precipitate was resuspended with 200 μl of FACS buffer, and the CAR expression rate on the surface of T cells was analyzed by flow cytometry.

### 4) Detection Method of Secreted IFN-γ Content

CAR-T cells and Nalm6 cells were inoculated into a 24-well plate at a ratio of 1:1 with 0.5×10⁶ cells/well as experimental well. Meanwhile, CAR-T cell control well and Nalm6 cell control well were set up. The 24-well plate was cultured in carbon dioxide incubator at 37° C., 5% CO₂ for about 24 hours.

The centrifugation was performed and the supernatant was collected after 24 hours of culture.

The microplate (IFN-γ Microplate) from sealed bag that has been balanced to room temperature was taken out, the unused strip was put back into aluminum foil bag, re-sealed and stored at 2-8° C.

The samples, detection reference and negative control were diluted 100 times with diluent (1×), respectively.

The prepared standards (concentration from low to high), samples, detection reference and negative control were added into corresponding wells respectively with 100 μl/well, and three repeat wells were set up. The reaction wells were sealed with sealing tape and incubated at room temperature for 2 hours.

The liquid in the plate was thrown off, the automatic washing machine was used, washing working solution at 350 μl/well was added and the plate was washed for 5 s at low-speed vibration, repeated 4 times; or the plate was washed manually, washing working solution at 300 μl/well was added, soaked for 30 s, repeated 4 times.

IFN-γ conjugate was added at 200 μl/well, sealed with sealing film and incubated at room temperature for 2 hours.

Step 4.7.5 was repeated to wash the plate.

Color developing solution was added at 2 μl/well, the plate was left to stand at room temperature and incubated for 10-30 min away from light.

Stop solution 1 was added at 50 μl/well, shaken gently and mixed.

The reading was detected with enzyme-labeled instrument at detection wavelength of 450 nm and reference wavelength of 570 nm.

### Plotting and Calculation of Standard Curve

4-parameters linear standard curve was plotted with enzyme-labeled instrument software. Horizontal coordinate of the curve is IFN-γ concentration value of the standard curve point, and vertical coordinate is average OD of the standard curve point (OD=OD₄₅₀-OD₅₇₀). The concentration of IFN-γ in samples can be obtained from the standard curve by using average OD of each sample.

### Example 1: Screening of Serum-Free Culture Medium in CAR-T Cell Culture Process

### Step 1: Obtaining T Cells

Peripheral blood mononuclear cells (PBMC) were separated from apheresis components of a subject by using glucan-meglumine diatrizoate (Ficoll) density gradient centrifugation method, then PBMC cells were sorted to obtain T cells by using magnetic activated cell sorting. The step of separating PBMC cells from apheresis components of a subject by using Ficoll density gradient centrifugation method was as follows:
(1) 2 ml of venous blood was taken and added into a test tube containing heparin solution (10-50 μg/ml of blood sample) and mixed in order to make the blood anticoagulant. The anticoagulant blood was diluted by 1 fold with pH 7.2 Hanks or saline.
(2) 2 ml of lymphocyte stratification solution was absorbed and placed into graduated centrifuge tube, then the centrifuge tube was tilted at a 45°, the diluted whole blood was added slowly along the tube wall to separation liquid with a capillary dropper. Attention should be paid to keep the interface clear.
(3) At 18° C.-20° C., centrifugation was performed with horizontal centrifuge at 2000 r/min for 20 min.
(4) The capillary straw was gently inserted into turbidity zone and the layer of cells was gently sucked out along the tube wall, then moved into another centrifugal tube. It is necessary to absorb all mononuclear cells and to avoid absorbing too much stratification solution or plasma, so as not to mix with other cellular components.
(5) The cells were washed with Hanks solution for 3 times: 2000 r/min, 10 min for the first time and 1500 r/min, 10 min for the 2-3 times, which could remove most of the mixed platelets.
(6) The precipitated cells (i.e., PMBC cells) were suspended in the culture medium for use.

The step of sorting PBMC cells to obtain T cells by using CD3 immunomagnetic beads (commercially available from Miltenyi Company) was as follows:
appropriate amount of MACS buffer (component: PBS/EDTA+0.2% BSA) was taken to wash PMBC cells (10⁷/mL). After centrifugation, PMBC was suspend with MACS buffer, then CD3 immunomagnetic beads (20 μL/10⁷ PBMC) were added and mixed, then incubated at 4° C. for 15 min. After washing the cells once with MACS buffer, the cells were suspended with 500 μL. The MS separation column was placed into magnetic field and pre-washed once with MACS buffer. The cell suspension was added to MS column, and the cells that flow out firstly were CD3T cells. The isolated cells were washed with MACS buffer for 3 times, the MS column was removed from magnetic field, then 1 mL of MACS buffer was added, and CD3T cells were pushed out into a sterile centrifuge tube with a push rod. After cell counting, the cells were divided into four parts and four different CAR-T cell complete culture medium, respectively: KBM581+5% FBS+100 IU/ml IL-2, X-VIVO+5% ISR+100 IU/ml IL-2, PRIME-XVT CELL CDM+5% ISR+100 IU/ml IL-2, AIM-V+5% ISR+100 IU/ml IL-2 were used to resuspend the cells, and the supernatant was removed by centrifugation at 1500 rpm for 10 minutes.

### Step 2: Carrying Out Activation Treatment on T Cells

The isolated T cells were resuspended with four CAR-T cell complete culture medium (KBM581+5% FBS+100 IU/ml IL-2, X-VIVO+5% ISR+100 IU/ml IL-2, PRIME-XVT CELL CDM+5% ISR+100 IU/ml IL-2, AIM-V+5% ISR+100 IU/ml IL-2) to a final concentration of 2×10⁶ cells/ml, and 2.5 μL of CD3/CD28 antibodies were added to stimulate magnetic beads for each 1×10⁶ T cells and mixed, then cultured in incubator at 37° C.+5% CO₂ for at least 24 hours.

Step 3: Infecting T Cells with Lentiviral Vector

The activated cultured T cells were taken out, polybrene with a final concentration of 8 μg/ml was added and mixed, and lentiviral vectors were added slowly with MOI=1. After mixing, the cell culture plate was placed into a centrifuge, centrifuged at 1500 rpm for 1.5 hours, then cultured in incubator at 37° C.+5% CO₂ for at least 24 hours.

Step 4: Expansion Culture of CAR-T Cells after Infection

After 24 hours of culture, the cell culture plate was centrifuged and the culture medium was discarded, then fresh cell culture medium was added to expand the CAR-T cells. Four different compositions of culture medium as mentioned above were used to expand the CAR-T cells and the cell culture medium was collected on day 0, day 2, day 4, day 6, day 8, day 10, day 12 and day 14 for the determination of expansion fold, survival rate and CAR expression rate.

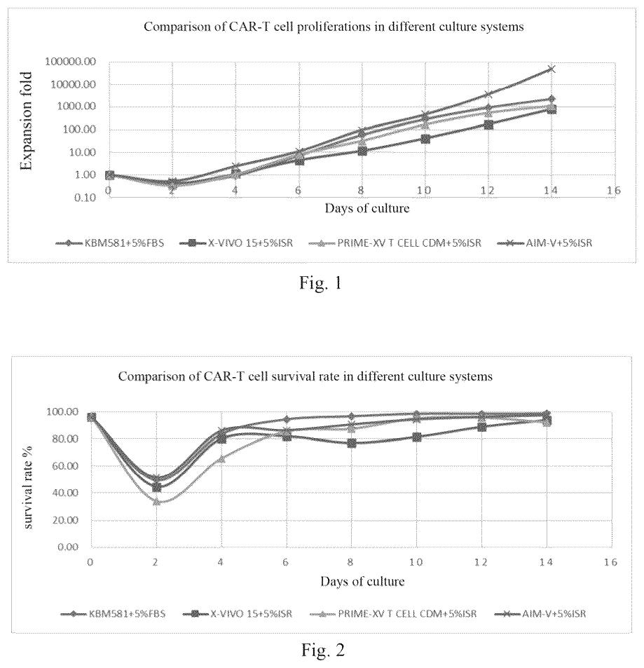

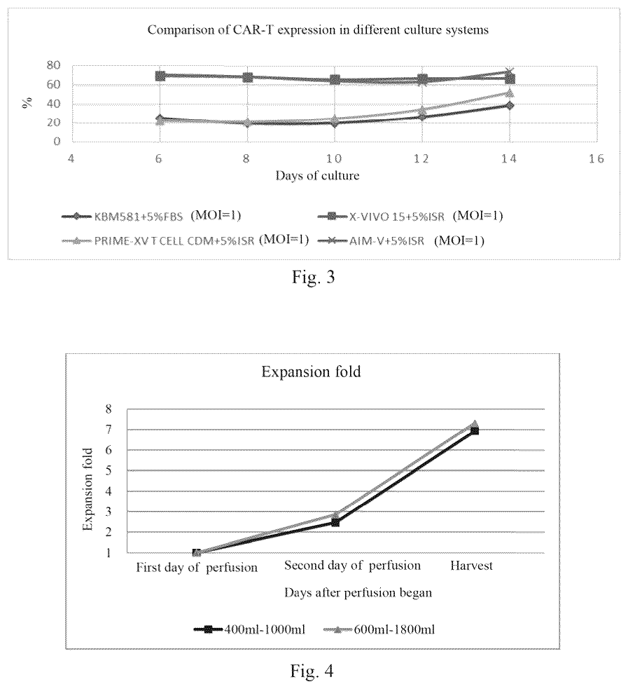

The results of expansion fold, survival rate, and CAR expression were shown in FIGS. 1-3. As can be seen from in FIGS. 1-3, in AIM-V+5% ISR+100 IU/mL IL-2 culture medium, CAR-T cell expansion fold, CAR-T cell survival rate and CD3⁺CAR⁺ expression were significantly better.

Based on the comparison of four kinds of culture medium and considering the culture effect, comprehensively, AIM-V+5% ISR culture medium was selected. Therefore, in the subsequent experiments, AIM-V+5% ISR+100 IU/mL IL-2 culture medium was selected as the culture medium for CAR-T cell expansion, and MOI=1 was selected for lentiviral vector transfection.

### Example 2: Determination of Perfusion Rate in CAR-T Cell Culture Process

### Step 1: Obtaining T Cells

Peripheral blood mononuclear cells (PBMC) were separated from apheresis components of a subject by using glucan-meglumine diatrizoate (Ficoll) density gradient centrifugation method, then PBMC cells were sorted to obtain T cells by using immunomagnetic bead method. The step of separating PBMC cells from apheresis components of a subject by using Ficoll density gradient centrifugation method was as follows:
(1) 2 ml of blood was taken intravenously and added into a test tube containing heparin solution (10-50 μg/ml of blood sample) and mixed in order to make the blood anticoagulant. The anticoagulant blood was diluted by 1 fold with pH 7.2 Hanks or saline.
(2) 2 ml of lymphocyte stratification solution was absorbed and placed into graduated centrifuge tube, then the centrifuge tube was tilted at a 45°, the diluted whole blood was added slowly along the tube wall to separation liquid with a capillary dropper. Attention should be paid to keep the interface clear.
(3) At 18° C.−20° C., centrifugation was performed with horizontal centrifuge at 2000 r/min for 20 min.
(4) The capillary straw was gently inserted into turbidity zone and the layer of cells was gently sucked out along the tube wall, then moved in another centrifugal tube. It is necessary to absorb all mononuclear cells and to avoid absorbing too much stratification solution or plasma, so as not to mix with other cellular components.
(5) The cells were washed with Hanks solution for 3 times: 2000 r/min, 10 min for the first time and 1500 r/min, 10 min for the 2-3 times, which could remove most of the mixed platelets.
(6) The precipitated cells (i.e., PMBC cells) were suspended in the culture medium for use.

The step of sorting PBMC cells to obtain T cells by using CD3 immunomagnetic beads (commercially available from Miltenyi Company) was as follows:
appropriate amount of MACS buffer (component: PBS/EDTA+0.5% Human Albumin) was taken to wash PMBC cells (10⁷/mL). After centrifugation, PMBC was suspend with MACS buffer, then CD3 immunomagnetic beads (20 μL/10⁷ PBMC) were added and mixed, then incubated at 4° C. for 15 min. After washing the cells once with MACS buffer, the cells were suspended with 500 μL. The MS separation column was placed into magnetic field and pre-washed once with MACS buffer. The cell suspension was added to MS column, and the cells that flow out firstly were CD3T cells. The isolated cells were washed with MACS buffer for 3 times, the MS column was removed from magnetic field, then 1 mL of MACS buffer was added, and CD3T cells were pushed out into a sterile centrifuge tube with a push rod. After cell counting, the cells were resuspended with AIM-V45% ISR+100 IU/ml IL-2 culture medium.

### Step 2: Carrying Out Activation Treatment on T Cells

The isolated T cells were resuspended with AIM-V+5% ISR+100 IU/ml IL-2 culture medium to a final concentration of 2-10⁶ cells/ml, and 2.5 μL of CD3/CD28 antibodies were added to stimulate magnetic beads for each 1×10⁶ T cells and mixed, then cultured in incubator at 37° C.+5% CO₂ for at least 24 hours.

Step 3: Infecting T Cells with Lentiviral Vector

The activated cultured T cells were taken out, polybrene with a final concentration of 8 μg/ml was added and mixed, and lentiviral vectors were added slowly with MOI=1. After mixing, the cell culture plate was placed into a centrifuge, centrifuged at 1500 rpm for 1.5 hours, then cultured in incubator at 37° C.+5% CO₂ for at least 24 hours.

Step 4: Expansion Culture after Transferring to Xuri Bioreactor

After 24 hours of culture, the cell culture plate was centrifuged and the culture medium was discarded, then fresh cell culture medium AIM-V+5% ISR+100 IU/mL IL-2 was added to expand the CAR-T cells. The number of cells was monitored, and when the number of cells reached (5-15)×10⁷, the cells were transferred to Xuri bioreactor for expansion culture.

After the expansion culture began, samples were collected and counted every day, and the fluid was fed-batch cultured according to the standard of (0.3-1)×10⁶ cells/mL density, the ventilation volume was set to (0.1-1) L/min, the rotational speed was (4-12) rpm, and the ventilation was compressed air plus 5% CO₂. When the culture volume reached 1000 mL and the total number of cells is ≥5×10⁸, and the cells were transferred to perfusion culture mode. After the perfusion culture began, the ventilation volume was set to 0.5 L/min, the rotational speed was 10 rpm, and the ventilation was compressed air plus 5% CO₂.

The expansion culture of CAR-T cells was performed by using different perfusion rate, and the data of expansion fold, survival rate and secreted IFN-γ content and the like after perfusion was compared. Four perfusion modes were compared, and the four perfusion modes were as follows, respectively:
(1) After the culture volume reached 1000 mL, when the cell density was (0.5-1.1)×10⁶ cells/mL, the perfusion volume was set to 400 mL/day; when the cell density was ≥2×10⁶ cells/mL, the perfusion volume was set to 1000 mL/day (hereinafter referred to as “400 mL-1000 mL mode”).
(2) After the culture volume reached 1000 mL, when the cell density was (1.1-2)×10⁶ cells/mL, the perfusion volume was set to 800 mL/day; when the cell density was ≥2×10⁶ cells/mL, the perfusion volume was set to 1000 mL/day (hereinafter referred to as “800 mL-1000 mL mode”).
(3) After the culture volume reached 1000 mL, when the cell density was (0.5-1.1)×10⁶ cells/mL, the perfusion volume was set to 600 mL/day; when the cell density was ≥2×10⁶ cell/mL, the perfusion volume was set to 1800 mL/day (hereinafter referred to as “600 mL-1800 mL mode”).
(4) After the culture volume reached 1000 mL, when the cell density was (1.1-2)×10⁶ cells/mL, the perfusion volume was set to 1000 mL/day; when the cell density was ≥2×10⁶ cells/mL, the perfusion volume was set to 1500 mL/day (hereinafter referred to as “1000 mL-1500 mL mode”).

### Research Results:

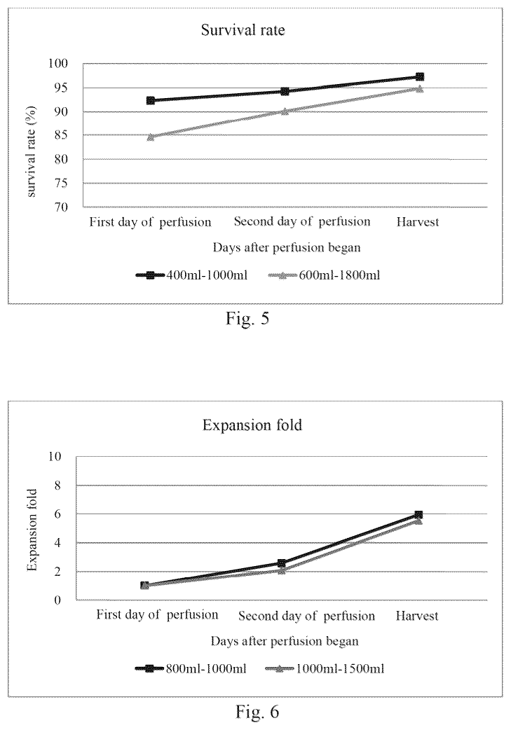

\textcircled{1} The results of cell density, survival rate, 24-hour expansion fold and secreted IFN-γ content after perfusion began (comparison between 400 mL-1000 mL mode and 600 mL-180 mL mode) were shown in Table 1 and FIGS. 4-5.

TABLE 1
cell density, survival rate, expansion fold and secreted IFN-γ content

| Perfusion rate: 400 mL-1000 mL | | | | | Perfusion rate: 600 mL-1800 mL | | | | |
|---|---|---|---|---|---|---|---|---|---|
| | | | | Secreted | | | | | Secreted |
| | | | | IFN-γ | | | | | IFN-γ |
| Perfusion | Cell density | Survival | Expansion | content | Perfusion | Cell density | Survival | Expansion | content |
| parameters | (×10⁶/mL) | rate (%) | fold | (pg/ml) | parameters | (×10⁶/mL) | rate (%) | fold | (pg/ml) |
| First day | 1.05 | 92.3 | 1 | — | First day of | 1.07 | 84.6 | 1 | — |
| of perfusion: | | | | | perfusion: | | | | |
| perfusion | | | | | perfusion | | | | |
| 400 mL | | | | | 600 mL | | | | |
| Second day | 2.57 | 94.2 | 2.46 | — | Second day | 3.05 | 90.1 | 2.85 | — |
| of perfusion: | | | | | of perfusion: | | | | |
| perfusion | | | | | perfusion | | | | |
| 1000 mL | | | | | 1800 mL | | | | |
| Third day | 7.22 | 97.2 | 6.91 | 84927.709 | This day of | 7.83 | 94.8 | 7.31 | 69795.842 |
| of perfusion: | | | | | perfusion: | | | | |
| harvest | | | | | harvest | | | | |

From the above results, it can be concluded that in the comparison of 400 mL-1000 mL mode and 600 mL-1800 mL mode, the cell expansion fold of 600 mL-1800 mL mode was slightly better than that of 400 mL-100 mL mode, while the survival rate and the secreted IFN-γ content of 400 mL-1000 mL mode were significantly higher than those of 600 mL-1800 mL mode, and the secreted IFN-γ content of 400 mL-1000 mL mode was about 1.2 times of that of 600 mL-1800 mL mode.

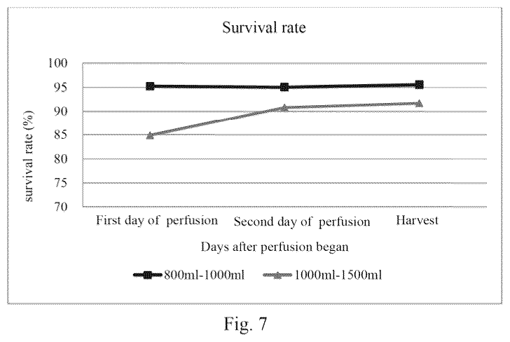

\textcircled{2} The results of cell density, survival rate, 24-hour expansion fold and secreted IFN-γ content after perfusion began (comparison between 800 mL-1000 mL mode and 1000 mL-1500 mL mode) were shown in Table 2 and FIGS. 6-7.

TABLE 2
cell density, survival rate, expansion fold and secreted IFN-γ content

| The first experiment: 800 mL-1000 mL | | | | | The first experiment: 1000 mL-1500 mL | | | | |
|---|---|---|---|---|---|---|---|---|---|
| | | | | Secreted | | | | | Secreted |
| | | | | IFN-γ | | | | | IFN-γ |
| Perfusion | Cell density | Survival | Expansion | content | Perfusion | Cell density | Survival | Expansion | content |
| parameters | (×10⁶/mL) | rate (%) | fold | (pg/ml) | parameters | (×10⁶/mL) | rate (%) | fold | (pg/m) |
| First day of | 1.77 | 95.2 | 1 | — | First day of | 1.56 | 84.87 | 1 | — |
| perfusion: | | | | | perfusion: | | | | |
| perfusion | | | | | perfusion | | | | |
| 800 mL | | | | | 1000 mL | | | | |
| Second day of | 4.59 | 95 | 2.59 | — | Second day | 3.24 | 90.8 | 2.08 | — |
| perfusion: | | | | | of perfusion: | | | | |
| perfusion | | | | | perfusion | | | | |
| 1000 mL | | | | | 1500 mL | | | | |
| Third day of | 10.5 | 95.5 | 5.93 | 61690.708 | Third day of | 8.61 | 91.7 | 5.52 | 34546.4 |
| perfusion: | | | | | perfusion: | | | | |
| harvest | | | | | harvest | | | | |

From the above results, it can be concluded that in the comparison of 800 mL-1000 mL mode and 1000 ml-1500 mL mode, there was no significant difference in the average results of cell expansion fold and survival rate between the two experiments, but the secreted IFN-γ content in 800 mL-1000 mL mode was significantly higher than that of 1000 mL-1500 mL mode, and the secreted IFN-γ content of 800 mL-1000 mL mode was about 1.8 times of that of 1000 mL-1500 mL mode.

Through the above experiments, the four perfusion modes were compared. Considering the culture effect and economy comprehensively, 400 ml-1000 ml or 800 mL-1000 mL perfusion mode was selected as the preferred perfusion mode, i.e., when the cell density is (0.5-1.1)×10⁶ cells/mL, the perfusion volume is set to 400 mL/day and/or, when the cell density is (0.1-2)×10⁶ cells/mL, the perfusion volume is set to 800 mL/day; and when the cell density is ≥2×10⁶ cells/mL, the perfusion volume is set to 1000 mL/day.

In conclusion, in AIM-V+5% ISR culture medium, CAR-T cells with high cell expansion fold, high cell survival rate, high secreted IFN-γ content, and guaranteed CAR⁺ expression can be obtained by perfusion mode of 400 mL-1000 mL or 800 mL-1000 mL, which can be used for clinical treatment.

1. A perfusion culture method for CAR-T cells, which comprises the following steps:
1) separating peripheral blood mononuclear cells from apheresis components of a subject, and sorting the peripheral blood mononuclear cells to obtain T cells;
2) carrying out activation treatment on the separated T cells with CD3/CD28 stimulation magnetic beads;
3) infecting the activated T cells with a lentiviral vector;
4) carrying out perfusion culture of the lentivirus-infected T cells, and harvesting CAR-T cells;
wherein a serum-free culture medium which does not contain an animal-derived component is used for culturing the CAR-T cell and the composition of the serum-free culture medium is: AIM-V+(3-9)% ISR;
the perfusion culture comprises the follow stages:
the first stage: when the cell density is 0.5-1.1×10⁶ cells/mL, the perfusion rate is A₁, and/or
the second stage: when the cell density is 1.1-2×10⁶ cells/mL, the perfusion rate is A₂, and
the third stage: when the cell density is >2×10⁶ cells/mL, the perfusion rate is A₃, wherein the ratio of A₁ to A₂ to A₃ is 1:2:2.5.

2. The perfusion culture method for CAR-T cells according to claim 1, wherein the composition of the serum-free culture medium which does not contain an animal-derived component is: AIM-V+(4-7)% ISR.

3. The perfusion culture method for CAR-T cells according to claim 1, wherein A₁ is 0.4 bioreactor volume/day, A₂ is 0.8 bioreactor volume/day, and A₃ is 1.0 bioreactor volume/day.

4. The perfusion culture method for CAR-T cells according to claim 1, wherein in step 4), the perfusion culture is carried out when the cell density is greater than or equal to a preset value.

5. The perfusion culture method for CAR-T cells according to claim 4, wherein in step 4), fed-batch culture is used before the cell density reaches the preset value, and in the process of fed-batch culture, the density of 0.3-1×10⁶ cells/mL is used as the standard for feeding, the ventilation volume is 0.1-1 L/min, the rotational speed is 4-12 rpm, and the ventilation is compressed air plus 1-10% CO₂.

6. The perfusion culture method for CAR-T cells according to claim 5, wherein in step 4), before the fed-batch culture, the method comprises: transferring the infected T cells to Xuri bioreactor for fed-batch culture after the number of infected T cells reaches 5-15×10⁷ cells.

7. The perfusion culture method for CAR-T cells according to claim 1, wherein in the process of perfusion culture, the ventilation volume is 0.3-0.8 L/min, the rotational speed is 5-rpm, and the ventilation is compressed air plus 1-10% CO₂.

8. The perfusion culture method for CAR-T cells according to claim 1, wherein in step 2), the step of carrying out activation treatment on the separated T cells with CD3/CD28 stimulation magnetic beads comprises: resuspending the isolated T cells to a final concentration of 1-2×10⁶ cells/mL, and mixing the T cells with CD3/CD28 stimulation magnetic beads at a ratio of 0.5-10 UL of CD3/CD28 magnetic beads per 1×10⁶ T cell, then culturing at 37° C.+5% CO₂ for at least 24 h.

9. The perfusion culture method for CAR-T cells according to claim 8, wherein the isolated T cells are resuspended with the serum-free culture medium which does not contain an animal-derived component, wherein the composition of the serum-free culture medium which does not contain an animal-derived component is: AIM-V+(3-9)% ISR.

10. The perfusion culture method for CAR-T cells according to claim 1, wherein in step 3), the step of infecting the activated T cells with a lentiviral vector comprises: taking out the activated and cultured T cells, adding polybrene with a final concentration of 5-10 μg/mL and mixing, then adding lentiviral vectors slowly at a multiplicity of infection of 0.25-5; mixing and then centrifuging at 1000-3000 rpm for 0.5-2.0 hours, then culturing at 37° C.+5% CO₂ for at least 24 hours.

11. The perfusion culture method for CAR-T cells according to claim 1, wherein the composition of the serum-free culture medium which does not contain an animal-derived component is: AIM-V+5% ISR.

12. The perfusion culture method for CAR-T cells according to claim 4, wherein the preset value is 0.3-1.2×10⁶ cells/mL.

13. The perfusion culture method for CAR-T cells according to claim 4, wherein the preset value is 0.4-1.0×10⁶ cells/mL.

14. The perfusion culture method for CAR-T cells according to claim 4, wherein the preset value is 0.5×10⁶ cells/mL.

15. The perfusion culture method for CAR-T cells according to claim 7, wherein in the process of perfusion culture, the ventilation volume is 0.4-0.6 L/min, the rotational speed is 8-12 rpm, and the ventilation is compressed air plus (3-6) % CO₂.

16. The perfusion culture method for CAR-T cells according to claim 7, wherein in the process of perfusion culture, the ventilation volume is 0.5 L/min, the rotational speed is 10 rpm, and the ventilation is compressed air plus 5% CO₂.

17. The perfusion culture method for CAR-T cells according to claim 8, wherein the isolated T cells are resuspended with the serum-free culture medium which does not contain an animal-derived component, wherein the composition of the serum-free culture medium which does not contain an animal-derived component is: AIM-V+(4-7)% ISR.

18. The perfusion culture method for CAR-T cells according to claim 8, wherein the isolated T cells are resuspended with the serum-free culture medium which does not contain an animal-derived component, wherein the composition of the serum-free culture medium which does not contain an animal-derived component is: AIM-V+5% ISR.
